(19) United States
(12) Patent Application Publication
(10) Pub. No.: US 2026/0057968 A1
(43) Pub. Date: Feb. 26, 2026

(54) Genome Characterisation System and Method

(71) Applicant: Kromek Limited, Sedgefield (GB)

(72) Inventors: Peter Sebastian Greenwood Rhodes, Sedgefield (GB); Ra'ad Munir David Mahmoud, Sedgefield (GB)

(21) Appl. No.: 19/130,798

(22) PCT Filed: Nov. 16, 2023
(86) PCT No.: PCT/GB2023/053018

§ 371 Date: May 16, 2025

(30) Foreign Application Priority Data
Nov. 17, 2022 (GB) 2217241.5
Nov. 17, 2022 (GB) 2217242.3

(51) Int. Cl. G16B 40/20; G06N 3/0464; G06N 3/048; G16B 30/00

(52) U.S. Cl. CPC G16B 40/20; G06N 3/0464; G06N 3/048; G16B 30/00

(57) ABSTRACT

A genome characterisation system for providing a genome characteristic prediction of a genome of origin associated with an input genomic sequence, the genome characterisation system comprising: an input preparation layer arranged to encode the input genomic sequence in a form suitable for input to a convolutional neural network; a multi-path residual block comprising a plurality of parallel residual routes, each residual route being adapted to receive input data from the input preparation layer and generate residual data corresponding to features of differing length; a self-attention layer arranged to receive residual data from each of the residual routes, generate a set of attention weights based on the residual data and a set of weights, and apply the set of attention weights to the residual data to generate an output tensor comprising data indicative of a relative importance of one or more portions of the input genomic sequence; and an output layer arranged to receive the output tensor from the self-attention layer; and output a likelihood vector indicative of characteristics of the genome of origin.

### FIELD OF THE INVENTION

The present invention relates to a neural network and/or a method of using a neural network to detect, classify, and/or characterise a genome.

### BACKGROUND TO THE INVENTION

The COVID-19 pandemic has shown that novel viruses with airborne transmission can have a catastrophic health, social and economic impact across the world. The rapid spread of COVID-19, Monkeypox, and other zoonoses have shown how animal diseases of minimal concern can quickly mutate to cause disease on a societal scale.

Existing genome classification systems may be based on sequence-alignment metrics (comparing genomic reads to reference genomes) and gene homology metrics (comparing genomic reads to viral gene databases). These reference-based approaches may be limited by the richness and reliability of reference databases. Furthermore, there may be no reliable way of identifying the emergence of novel phenotypic or genotypic traits, even when those traits have been observed in other taxonomic groups.

Other existing genome classification systems may use alignment-free k-mer based metrics. Such a system may be purely k-mer based. Thus, such a system may lack sufficient structural or contextual information in the sequence read beyond the k-mer scale, which may be in the order of a few bases.

The present invention has been devised to address at least some of the aforementioned problems.

### SUMMARY OF THE INVENTION

In accordance with a first aspect of the present disclosure, there is provided a genome characterisation system for providing a genome characteristic prediction of a genome of origin associated with an input genomic sequence, the genome characterisation system comprising: an input preparation layer arranged to encode the input genomic sequence in a form suitable for input to a convolutional neural network; a multi-path residual block comprising a plurality of parallel residual routes, each residual route being adapted to receive input data from the input preparation layer and generate residual data corresponding to features of differing length; a self-attention layer arranged to receive residual data from each of the residual routes, generate a set of attention weights based on the residual data and a set of weights, and apply the set of attention weights to the residual data to generate an output tensor comprising data indicative of a relative importance of one or more portions of the input genomic sequence; and an output layer arranged to receive the output tensor from the self-attention layer; and output a likelihood vector indicative of characteristics of the genome of origin.

The ‘genome of origin’ may be understood as a parent genome from which the input genomic sequence originates. For example, the parent genome may be a SARS-COV-2 genome, and the input genomic sequence may be a portion of the SARS-COV-2 genome.

The input preparation layer may provide a means for the genome characterisation system to interface with an input genomic sequence, by converting the input genomic sequence, which may be a linear sequence of nucleotides, to a form that is suitable for a convolutional neural network.

The multi-path residual block may provide a means for extracting information from the input genomic sequence relating to different features, wherein each feature is a k-mer, of differing length. This information may improve a characterisation performance of the system.

The self-attention layer may provide a means for increasing the influence of some features (i.e., k-mers) over other features. The self-attention layer may be a superior alternative to solely concatenating the outputs of the residual routes together. In particular, the self-attention layer may allow the neural network to effectively weight (i.e., pay more attention) to those features (or combinations of features) from all residual route outputs that are more important for characterisation of the input genomic sequence. Without the self-attention layer, each output feature map from each residual route would have an equal weighting over the result, regardless of the overall set of extracted features. This lack of feature preference can lead to a reduction in characterisation performance. Thus, the self-attention layer may provide an improved characterisation performance.

The genome characterisation system may provide a tool which can holistically understand information from across the entire read structure, and heuristically apply knowledge from all seen species to any new genomic read.

In some embodiments, the input preparation layer is arranged to encode the input genomic sequence as a multi-channel representation of the input genomic sequence. The multi-channel representation may advantageously provide a means for storing and conveying more information of the input genomic sequence. As such, an eventual genome characteristic prediction based on this multi-channel representation may be based on higher quality data, and thus predictions may be more accurate to ground truth labels.

In some embodiments, the input preparation layer is arranged to encode the input genomic sequence as the multi-channel representation by: receiving the input genomic sequence; generating a first channel comprising a frequency chaos game representation (FCGR) of the input genomic sequence; generating a second channel representing a syntactic sequential relationship between nucleotide encodings in the first channel; and encoding the input genomic sequence by combining the first and second channels to create the multi-channel representation of the input genomic sequence. The first channel may provide a visual representation of the linear sequence of nucleotides in the input genomic sequence. Such a visual representation may be more suitable for input to a CNN. The second channel may provide additional information on relations between nucleotides and/or k-mers that provides syntax of the input sequence. As such, the multi-channel representation may capture the sequential information between the nucleotides in a way that the standard CGR does not, thereby improving a classification accuracy.

In some embodiments, the input preparation layer is further arranged to: generate an input tensor representative of the input genomic sequence. Thus, the input preparation layer may prepare an input suitable for a CNN.

In some embodiments, the input tensor is a three-dimensional tensor comprising: a height and width representative of the spatial aspect of the encoded genomic sequence; and a depth representative of the multi-channel aspect of the encoded genomic sequence.

In some embodiments, each residual route comprises one or more residual blocks connected in sequence, each residual block comprising convolutional layers; wherein each convolutional layer of a residual route is of the same kernel size; and wherein the kernel size of convolutional layers in a first residual route is different to the kernel size of convolutional layers in a second residual route. Since the kernel size of convolutional layers of a first residual route is different to those of a second residual route, each residual route may extract information about k-mers of different lengths. The optimal k-mer length for processing genomic data is a subject of wide debate. The length of “k” can significantly influence analytical results. For example, a smaller “k” value may be suitable for smaller-scale features but may be insufficient for identifying larger-scale features. A larger “k” value may be suitable for identifying longer-scale features but may miss smaller-scale features. By using residual routes that comprise convolutional layers having different and unique kernel sizes, the multi-path residual block can examine the input tensor on a range of different scales. The multi-path residual block can thus address the optimal k-mer length problem when processing genomic data by processing a plurality of different k-mer lengths.

In some embodiments, the self-attention layer is arranged to: generate a concatenated input by concatenating the input data from the plurality of residual routes; generate a first transposed input tensor by transposing the concatenated input; generate a first intermediate tensor by multiplying the first transposed input tensor by a first weight tensor; generate a second intermediate tensor by applying a first activation function to the first intermediate tensor; generate a third intermediate tensor by multiplying the second intermediate tensor by a second weight tensor; re-shape the third intermediate tensor to a form suitable for input to a second activation function; generate a fourth intermediate tensor by applying the second activation function to the re-shaped third intermediate tensor; generate an attention tensor comprising the set of attention weights by reshaping a fourth intermediate tensor; generate a second transposed input tensor by transposing the concatenated input; generate an attention output by multiplying the second transposed input tensor by the attention tensor; and generate an output tensor by transposing the attention output.

In some embodiments, the output layer is arranged to: flatten the output tensor of the self-attention layer; input the flattened tensor to a densely connected layer; and apply a dense sigmoid activation function. The output layer may produce an output that can be used to characterize the genome of origin of the input genomic sequence.

In accordance with a second aspect of the present disclosure, there is provided: a genome characterisation method for providing a genome characteristic prediction of a genome of origin associated with an input genomic sequence, the genome characterisation method comprising: encoding the input genomic sequence in a form suitable for input to a convolutional neural network; generating residual data corresponding to features of differing length; generating a set of attention weights based on the residual data and a set of weights, applying the set of attention weights to the residual data to generate an attention output comprising data indicative of a relative importance of one or more portions of the input genomic sequence; and outputting a likelihood vector indicative of characteristics of the genome of origin.

In accordance with a third aspect of the present disclosure, there is provide a genomic sequence encoding method comprising: receiving an input genomic sequence, the input genomic sequence comprising a one-dimensional linear sequence of nucleotide bases representing a particular genome; and encoding the input genomic sequence in a form suitable for input to a convolutional neural network.

In some embodiments, the input genomic sequence is encoded as a multi-channel representation of the input genomic sequence. The multi-channel representation may advantageously provide a means for storing and conveying more information of the input genomic sequence. As such, an eventual genome characteristic prediction based on this multi-channel representation may be based on higher quality data, and thus predictions may be more accurate to ground truth labels.

In some embodiments, encoding the input genomic sequence as the multi-channel representation comprises: receiving the input genomic sequence; generating a first channel comprising a frequency chaos game representation (FCGR) of the input genomic sequence; generating a second channel representing a syntactic sequential relationship between nucleotide encodings in the first channel; and encoding the input genomic sequence by combining the first and second channels to create the multi-channel representation of the input genomic sequence. The first channel may provide a visual representation of the linear sequence of nucleotides in the input genomic sequence. Such a visual representation may be more suitable for input to a CNN. The second channel may provide additional information on relations between nucleotides and/or k-mers that provides syntax of the input sequence.

In accordance with a fourth aspect of the present disclosure, there is provided a method for training a genome characterisation neural network comprising: preparing a training dataset comprising a plurality of training genomic sequences; encoding the training genomic sequences in a form suitable for input to a convolutional neural network; inputting an encoded genomic sequence into the neural network; obtaining a final output of the neural network; updating parameters of the neural network based on the final output; and repeating the inputting, obtaining, and updating steps until a training threshold is met.

In some embodiments, preparing the training dataset comprises: obtaining reference genomes of a plurality of species; extracting one or more genomic sequences from each reference genome; modifying each of the genomic sequences; and labelling each modified genomic sequence with characteristic labels of the respective reference genome.

Extracting one or more genomic sequences from each reference genome may comprise: extracting one or more slices of the reference genome, each slice being a genomic sequence having a sequence length.

Modifying each genomic sequence may comprise applying an insertion, a deletion and/or a base flip to the genomic sequence. Applying insertions to the genomic sequence may comprise inserting one or more nucleotides into the genomic sequence. Applying deletions to the genomic sequence may comprise deleting one or more nucleotides from the genomic sequence. Applying base flips to the genomic sequence may comprise changing one or more nucleotides in the genomic sequence.

Regardless of the sequencing method used, genomic reads may be subject to errors from several subprocesses in the sequencing process, including library preparation (for example, attachment of primers and amplification), and the sequencing procedure itself (for example, in nanopore sequencing, the voltage signal can be misinterpreted by base-callers, resulting in insertions, deletions, and base-flips). Existing long-read sequencing methods may have an error rate ranging from 1% to 8%. Application of insertions, deletions, and base flips is preferably in proportions that substantially match real world data, to replicate real-world applications of the model more accurately. Thus, the training data provided to the neural network is of higher quality (i.e., more in line with data that will be input in a real-world application), leading to a neural network with improved classification capabilities.

Encoding the training genomic sequences in a form suitable for to the convolutional neural network may comprise: generating a first channel comprising a frequency chaos game representation of the input genomic sequence; generating a second channel representing a syntactic sequential relationship between nucleotides in the first channel; and encoding the input genomic sequence by combining the first and second channels to create a multi-channel representation.

The final output of the neural network may be a vector of length n, wherein n is the number of labels.

Updating parameters of the neural network based on the final output may comprise: comparing the final output of the neural network to ground truth values using a modified loss function.

In accordance with a fifth aspect of the present disclosure, there is provided a neural network for providing a genome characteristic prediction of a genome of origin associated with an input genomic sequence, the neural network comprising: a multi-path residual block comprising a plurality of parallel residual routes, each residual route being adapted to receive input data and generate residual data corresponding to features of differing length; a self-attention layer arranged to receive residual data from each of the residual routes, generate a set of attention weights based on the residual data and a set of weights, and apply the set of attention weights to the residual data to generate an attention output comprising data indicative of a relative importance of one or more portions of the input genomic sequence; and an output layer arranged to receive the an attention output from the self-attention layer; and output a likelihood vector indicative of characteristics of the genome of origin.

In some embodiments, the input data is a three-dimensional tensor comprising: a height and width representative of the spatial aspect of an encoded genomic sequence; and a depth representative of a multi-channel aspect of the encoded genomic sequence.

In some embodiments, each residual route comprises one or more residual blocks connected in sequence, each residual block comprising convolutional layers; wherein each convolutional layer of a residual route is of the same kernel size; and wherein the kernel size of convolutional layers in a first residual route is different to the kernel size of convolutional layers in a second residual route.

In some embodiments, the self-attention layer is arranged to: generate a concatenated input by concatenating the input data from the plurality of residual routes; generate a first transposed input tensor by transposing the concatenated input; generate a first intermediate tensor by multiplying the first transposed input tensor by a first weight tensor; generate a second intermediate tensor by applying a first activation function to the first intermediate tensor; generate a third intermediate tensor by multiplying the second intermediate tensor by a second weight tensor; re-shape the third intermediate tensor to a form suitable for input to a second activation function; generate a fourth intermediate tensor by applying the second activation function to the re-shaped third intermediate tensor; generate an attention tensor comprising the set of attention weights by reshaping a fourth intermediate tensor; generate a second transposed input tensor by transposing the concatenated input; generate an attention output by multiplying the second transposed input tensor by the attention tensor; and generate an output tensor by transposing the attention output.

In some embodiments, the output layer is arranged to: flatten the output tensor of the self-attention layer; input the flattened tensor to a densely connected layer; and apply a dense sigmoid activation function.

It will be appreciated that any features described herein as being suitable for incorporation into one or more aspects or embodiments of the present disclosure are intended to be generalisable across any and all aspects and embodiments of the present disclosure. Other aspects of the present disclosure can be understood by those skilled in the art in light of the description, the claims, and the drawings of the present disclosure. The foregoing general description and the following detailed description are exemplary and explanatory only and are not restrictive of the claims.

### BRIEF DESCRIPTION OF THE DRAWINGS

The invention will now be described by way of example with reference to the following Figures in which:

FIG. 1 shows a genome characterisation system in accordance with a first aspect of the present disclosure;

FIG. 2 shows a flow diagram of an example process for encoding a one-dimensional genomic sequence in a form suitable for input to a CNN;

FIG. 3 shows a standard chaos game representation (CGR) of the SARS COV-2 genomic sequence (left), and frequency chaos game representation (FCGR) of the SARS COV-2 genomic sequence (right);

FIG. 4 shows a schematic view of an example residual route;

FIG. 5 shows a schematic view of the architecture of a self-attention layer;

FIG. 6 shows a flow diagram of a self-attention method using the self-attention layer of FIG. 5;

FIG. 7 shows a genome characterisation method using the genome characterisation system of FIG. 1; and

FIG. 8 shows a flow diagram of a method for training a genome characterisation neural network of the genome characterisation system of FIG. 1.

### DETAILED DESCRIPTION

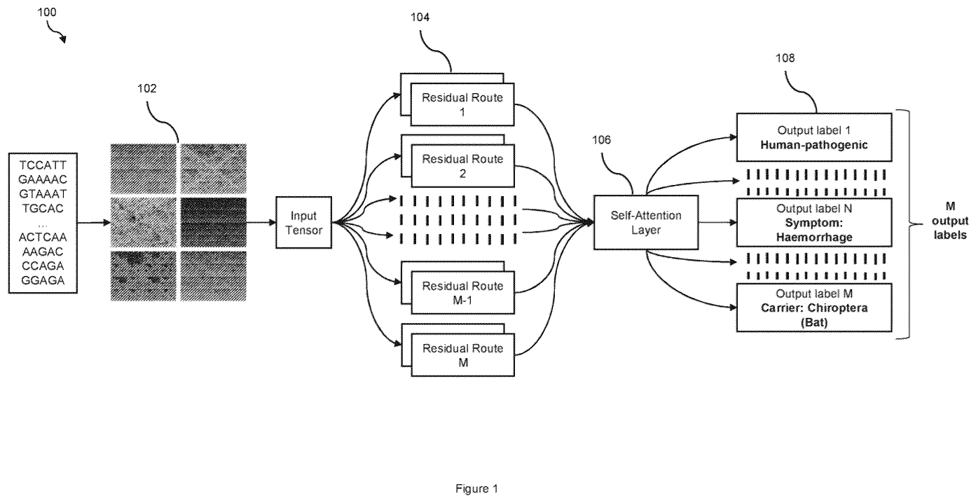

Referring to FIG. 1, there is shown a genome characterisation system 100. The genome characterisation system 100 comprises a neural network configured to provide a genome characteristic prediction of a genome of origin associated with an input genomic read.

The genome characterisation system 100 comprises: an input preparation layer 102; and a neural network comprising: a residual block 104; a self-attention layer 106; and an output layer 108.

Input preparation layer Inputs to neural networks must translate and preserve real features of the source data (in this case, genomic sequences) into a numerical format that is compatible with the neural network's structure, in particular a Convolutional Neural Network (CNN) structure. The present invention uses a two-layer input format based on chaos game representation (CGR). This format captures the k-mer frequency of a given genomic sequence using frequency chaos game representation (FCGR) in a first channel, while preserving the syntactic sequential relationship of each nucleotide via vectors generated using CGR in a second channel.

CGR nucleotide sequence encoding is described in Almeida J S, Carrico J A, MaretzekA, Noble P A, Fletcher M. Analysis of genomic sequences by Chaos Game Representation. Bioinformatics. 2001 May 1;17(5):429-37. Relevant methodologies are also described in Rizzo R, Fiannaca A, La Rosa M, Urso A. Classification experiments of DNA sequences by using a deep neural network and chaos game representation. In Proceedings of the 17th International Conference on Computer Systems and Technologies 2016 2016 Jun 23 (pp. 222-228).

To achieve this two-layer input format, the genome characterisation system 100 comprises an input preparation layer 102 arranged to encode an input genomic sequence in a form suitable for input to a neural network, more particularly a CNN. The input preparation layer 102 is arranged to encode the genomic sequence as a multi-channel representation of the input genomic sequence. In particular, the input preparation layer 102 is arranged to receive an input genomic sequence and encode the linear genomic sequence into a 2D space comprising two channels.

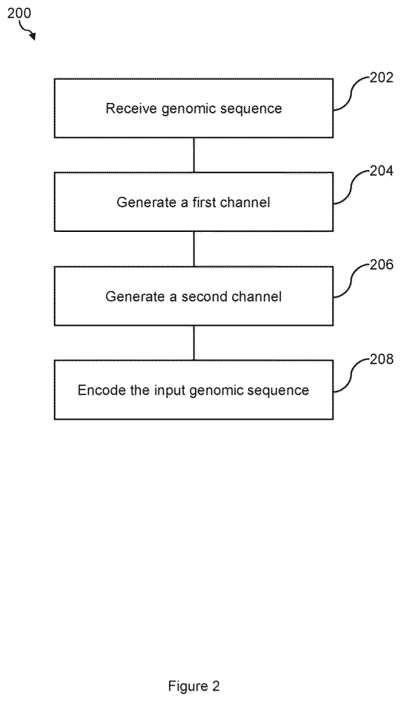

FIG. 2 shows a flow chart depicting an example process 200 for encoding a one-dimensional genomic sequence in a form suitable for input to a CNN. The process 200 uses CGR. CGR is an iterative method to project a continuous sequence of data onto a 2-D plot, such that each point is unique, and the original sequence is preserved.

In a first step 202 of the process 200, an input genomic sequence is received. The genomic sequence comprises a one-dimensional linear sequence of nucleotide bases representing a particular genome. An example genomic sequence is depicted in the left-most portion of FIG. 1.

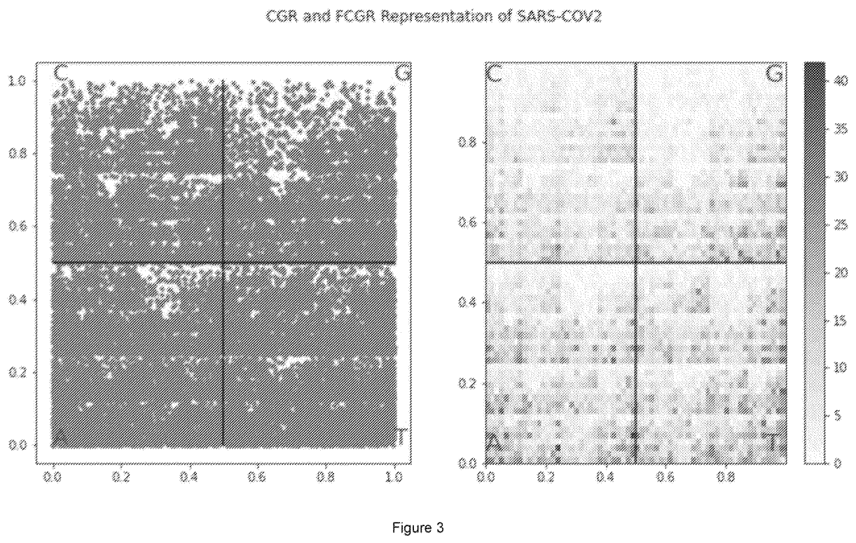

At step 204, a first channel is generated comprising a frequency chaos game representation (FCGR) of the input genomic sequence. To generate the FCGR, a unit square is generated (see FIG. 3), wherein each corner of the square is labelled according to a respective nucleotide base. A first corner (0,0) of the square is labelled as Adenine (A). A second corner (0,1) of the square is labelled as Cytosine (C). A third corner (1,1) of the square is labelled as Guanine (G). A fourth corner (1,0) of the square is labelled as Thymine (T). In other words, the coordinates of a current nucleotide (gᵢ) is gᵢ={A=(0, 0), T=(1, 0), C=(0, 1), G=(1, 1)}. An initial position of the unit sphere is plotted. The initial position is a central position (0.5, 0.5) of the square, although it could be elsewhere. Nucleotides points or coordinates are sequentially plotted according to Equation (1):

$$C G {R}_{i} = C G {R}_{i - 1} - 0.5 ( C G {R}_{i - 1} - {g}_{i} )$$

Wherein CGRᵢ is a next coordinate;
CGRᵢ₋₁ is the current coordinate; and
gᵢ is the coordinate of the next nucleotide in the genomic sequence.

For example, the current coordinate CGRᵢ₋₁ could be the central position (0.5, 0.5). The next nucleotide in the genomic sequence could be C, such that the nucleotide coordinate gᵢ is (0,1). Thus, the next coordinate CGR, is CGR, =(0.5, 0.5)−0.5((0.5, 0.5)−(0,1))=(0.25, 0.75). Thus, the next coordinate is halfway between the current coordinate and the coordinate of the next nucleotide in the genomic sequence.

The nucleotide points are plotted sequentially for each nucleotide in the genomic sequence, such that each nucleotide is fed through the equation in turn generating a coordinate in the unit square which can be mapped back to the specific nucleotides.

Equation 1 allows for two representations of the input genomic sequence. The first representation is the standard Chaos Game Representation (CGR), where CGR, denotes the CGR position of each nucleotide gᵢ of the input genomic sequence is placed in the unit square (see FIG. 3). In this encoding, the plot is aggregated over a

$${4}^{\frac{k}{2}} \times {4}^{\frac{k}{2}}$$

grid, to form a 2-dimensional histogram of k-mer distributions, the frequency chaos game representation (FCGR). The standard FCGR provides the first channel of the encoding by the input preparation layer 102, capturing the k-mer frequency of a given genomic sequence. FIG. 3 shows a standard chaos game representation (CGR) of the SARS COV-2 genomic sequence (left), and frequency chaos game representation (FCGR) of the SARS COV-2 genomic sequence (right).

At step 206, a second channel is generated. The second channel represents a syntactic relationship between nucleotide encodings in the first channel. The second channel is created from the vectors used to step between nucleotide encodings in the first channel. These vectors capture the sequential information between the nucleotides in a way that the standard CGR does not. More particularly, a plurality of vectors are generated, the vectors being the difference between the current nucleotide coordinate and the coordinate of the next nucleotide in the sequence, −0.5(CGRᵢ₋₁−gᵢ). The generated vectors are binned to create a 2D histogram representation. Continuing with the example of the next coordinate CGR, in the first channel being CGR, =(0.25, 0.85), a corresponding point in the second channel would be the difference portion of Equation 1, such that the corresponding second layer point is −0.5((0.5, 0.5)−(0,1))=(−0.25,0.25).

The second channel preserves the syntactic sequential relationship of each nucleotide through vectors generated using CGR during generation of the first channel.

At step 208, the input genomic sequence is encoded by combining the first and second channels to create the multi-channel representation of the input genomic sequence.

By encoding the input genomic sequence using the first channel and the second channel, more information may be captured. In particular, the first channel provides an FCGR representation that provides the content of the input genomic sequence, whilst the second channel provides relations between nucleotides and/or k-mers that provides syntax of the input sequence. The resultant multi-channel image is structured such that existing 2D convolutional methods can be applied for training with these encoded genomic sequences.

A first test was conducted in which a plurality of genomic sequences of 6 test species were encoded according to the FCGR representation only (i.e., a single layer encoding). A mean cost function value averaged across all 6 test species and 71 trait flags was 0.07. A second test was conducted in which the plurality of genomic sequences were encoded according to both the FCGR representation and the syntactic representation (i.e., the double-layer encoding of the present invention), A mean cost function value averaged across all 6 test species and 71 trait flags was 0.005. Thus, a 44% performance gain was achieved using the double-layer encoding of the present invention when compared with a single-layer encoding using the FCGR representation only.

The input preparation layer 102 is arranged to provide input data to the rest of the system 100. In particular, the input preparation layer 102 is arranged to generate an input tensor representative of the input genomic sequence. The input tensor is a three-dimensional tensor comprising a height and width representative of the spatial aspect of the encoded genomic sequence, and a depth representative of the multi-channel aspect of the encoded genomic sequence. The depth is due to the two-channel structure of the encoded genomic sequence. Thus, the input data is suitable for input to a convolutional neural network.

### Multi-Path Residual Block

The genome characterisation system 100 also comprises a multi-path residual block 104. The multi-path residual block 104 is arranged to receive input data from the input preparation layer 102.

The multi-path residual block 104 comprises a plurality of parallel residual routes 1 to M. Each residual route is adapted to receive input data from the input preparation layer (i.e., the input tensor) and generate residual data corresponding to features (i.e., k-mers) of differing length.

Each residual route comprises one or more residual blocks connected in sequence. Each residual block of a residual route comprising filters (i.e., convolutional layers) of the same kernel size. The kernel size of convolutional layers in each residual route is different to the kernel size of convolutional layers in the other residual routes. Each residual route has the same number of convolutional layers, such that data provided by the residual routes are compatible.

The optimal k-mer length for processing genomic data is a subject of wide debate. The length of “k” can significantly influence analytical results. For example, a smaller “k” value may be suitable for smaller-scale features but may be insufficient for identifying larger-scale features. A larger “k” value may be suitable for identifying longer-scale features but may miss smaller-scale features. By using residual routes that comprise convolutional layers having different and unique kernel sizes, the multi-path residual block 104 can examine the input tensor on a range of different scales. The multi-path residual block 104 can thus address the optimal k-mer length problem when processing genomic data by processing a plurality of different k-mer lengths.

Each residual route comprises other layers as is known in the art, such as batch normalisation layers, activation function layers, and dropout layers. Each residual route can also comprise bottleneck filters having a kernel size of 1×1.

Each residual route comprises convolutional filters of differing kernel size to the other residual routes. Residual route 1 comprises convolutional layers having a differing kernel size to residual route 2, which comprises convolutional layers having a differing kernel size to residual route M-1, and so on. Each convolutional filter is 3-dimensional, due to the input data being 3-dimensional. More particularly, each convolutional filter has a depth of 2, due to the input data being a two-channel image.

In an example implementation, residual route 1 comprises convolutional layers having a kernel size of 3×3, residual route 2 comprises convolutional layers having a kernel size of 4×4, residual route M-1 comprises convolutional layers having a kernel size of (M+1)×(M+1), and residual route M comprises convolutional layers having a kernel size of (M+2)×(M+2). It will be appreciated that these kernel sizes are by way example only, and that any suitable kernel size may be selected for each residual block. In a further example implementation, three residual routes are utilised, wherein a first residual route comprises convolutional layers having a kernel size of 3×3, a second residual route comprises convolutional layers having a kernel size of 5×5, and a third residual route comprises convolutional layers having a kernel size of 8×8. It will be understood that these example implementations may be modified, for example considering optimisation steps as training data and traits are added.

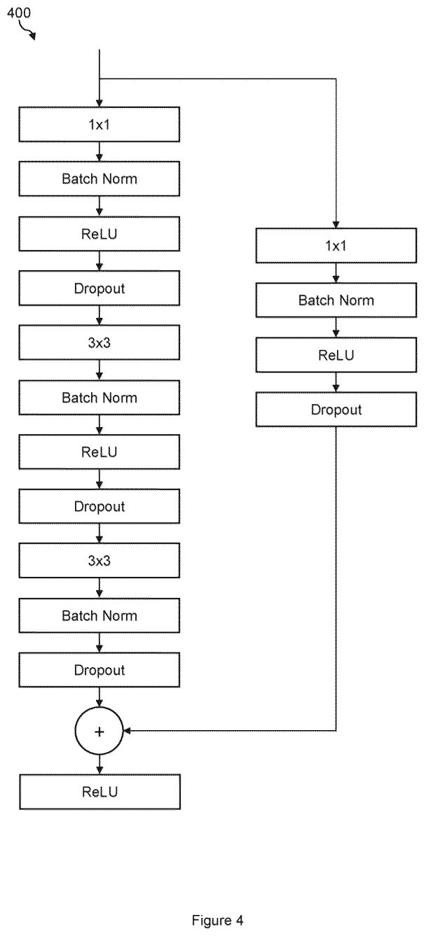

A schematic view of an example residual route 400 is shown in FIG. 4. The residual route 400 comprises convolution layer of kernel size 3×3. A main spine of the residual route 400 applies, to the input tensor, a 1×1 convolution, a batch normalisation, a ReLU activation function, a dropout, a 3×3 convolution, a batch normalisation, a ReLU activation function, a dropout, a 3×3 convolution, a batch normalisation, and a dropout. A shortcut route of the residual route 400 applies, to the input tensor, a 1×1 convolution, a batch normalisation, a ReLU activation function, and a dropout. The output of the shortcut route is added to the output of the main spine, and a ReLU activation function is applied.

The multi-path residual block 104 is arranged to provide the residual data input to the self-attention layer 106. In particular, the multi-path residual block 104 is arranged such that the residual data outputs of each of the residual routes 1 to M are provided to the self-attention layer 106. The self-attention layer 106 can contextualise the features extracted from each scale with one-another to build a better picture of the relations between large- and small-scale features.

### Self-Attention Layer

The genome characterisation system 100 comprises a self-attention layer 106. The self-attention layer 106 is arranged to receive residual data from each of the residual routes 1 to M, generate a set of attention weights based on the residual data and a set of weights, and apply the set of attention weights to the residual data to generate an output tensor comprising data indicative of a relative importance of one or more portions (i.e., k-mers) of the input genomic sequence.

The set of weights are optimised during a training process discussed further below. The set of attention weights are thus based on an optimised parameter, and the residual data that is dependent on the input genomic sequence. Therefore, the set of attention weights is unique to each input genomic sequence and the self-attention layer thus generates an attention output that is also unique to each input genomic sequence. Therefore, the importance of different k-mers of the input genomic sequence is unique to each input genomic sequence.

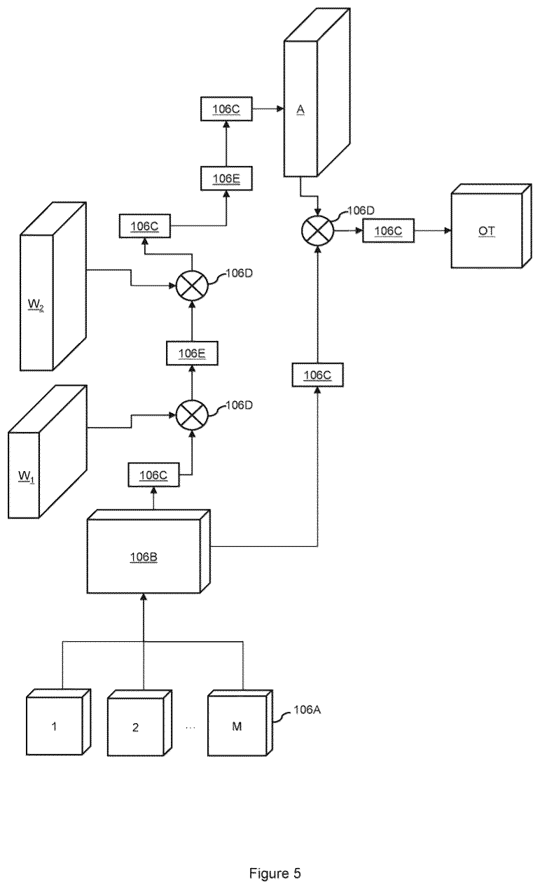

FIG. 5 shows a schematic view of the architecture of the self-attention layer 106. The self-attention layer 106 comprises an input layer 106A, a concatenation unit 106B, a tensor manipulation unit 106C, a tensor multiplication unit 106D, an activation function unit 106E, and an output layer 106F. The self-attention layer 106 also uses a first weight tensor W₁ and a second weight tensor W₂, both weight tensors comprising weights optimised during training of the neural network. The first weight tensor W₁ has dimensions X_{w1}, Y_{w1}, Z_{w1}. The second weight matrix W₂ has dimensions X_{w2}, Y_{w2}, Z_{w2}.

The input layer 106A is adapted to receive input data from a plurality of sources (i.e., from the residual blocks 1 to M). Each output of the residual blocks 1 to M comprises an X dimension, a Y dimension, and a Z dimension. The X and Y dimensions correspond to the X and Y dimensions of the input tensor. The Z dimension corresponds to the number of filters present in the respective residual blocks, since each filter produces one output channel.

The concatenation unit 106B is arranged to generate a concatenated input by concatenating the input data (i.e., from the residual blocks 1 to M). Accordingly, the concatenated input produced at the concatenation block comprises a first dimension of length NX, a second dimension of length Y, and a third dimension of a length corresponding to the number of filters.

The tensor manipulation unit 106C is arranged to transpose or reshape tensors. In particular, the tensor manipulation unit 106C is arranged to: generate a first transposed input tensor by transposing the concatenated input to a form suitable for tensor multiplication with the first weight tensor W₁; re-shape a third intermediate tensor to a form suitable for interaction with the self-attention tensor; and generate a second transposed input tensor by transposing the concatenated input to a form suitable for tensor multiplication with an attention tensor.

The tensor multiplication unit 106D is arranged to multiply tensors. In particular, the tensor multiplication unit 106D is arranged to: generate a first intermediate tensor by multiplying the first transposed input tensor with the first weight tensor W₁; and generate the third intermediate tensor by multiplying a second intermediate tensor by the second weight tensor W₂.

The activation function unit 106E is arranged to apply activation functions to inputs. In particular, the activation function unit 106E is arranged to: generate the second intermediate tensor by applying a first activation function to the first intermediate tensor; and generate a fourth intermediate tensor by applying a second activation function to the re-shaped third intermediate tensor.

The self-attention layer 106 uses a first weight matrix 106C having a plurality of weights W₁ and dimensions X_{w1}, Y_{w1}, Z_{w1}. The self-attention layer 106 further comprises a second weight matrix 106D having a plurality of weights W₂ and dimensions X_{w2}, Y_{w2}, Z_{w2}.

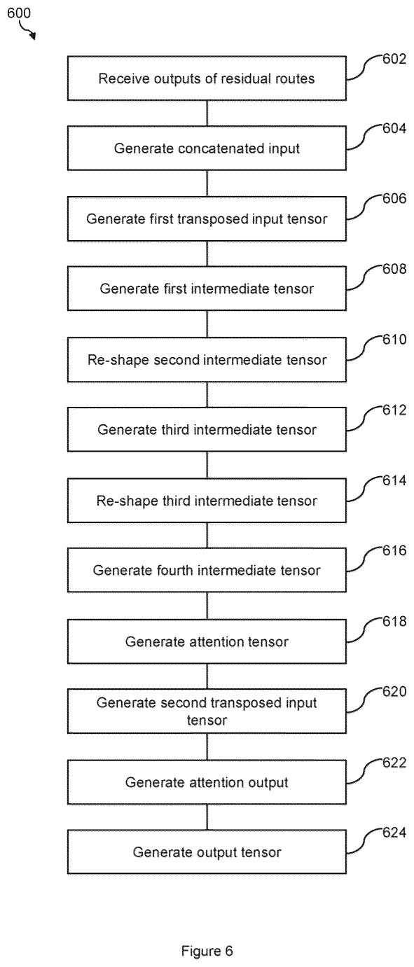

FIG. 6 shows a flow diagram of a self-attention method 600 using the self-attention layer 106.

In a first step 602, the input layer 106A receives the outputs of the residual blocks 1 to M. Each output of the residual routes 1 to M comprises an X dimension, a Y dimension, and a Z dimension. The X and Y dimensions correspond to the X and Y dimensions of the input tensor. The Z dimension corresponds to the number of filters present in the respective residual blocks, since each filter produces one output channel. The residual routes 1 to M each comprise the same numbers of filters, such that their outputs are compatible.

At step 604, the concatenation unit 106B generates a concatenated input H by concatenating the outputs of the residual routes 1 to M. Accordingly, a concatenated input H produced at the concatenation block comprises a first dimension (X) of length MX (wherein N is the number of filters in each residual route 1 to M), a second dimension (Y) of length Y, and a third dimension (Z) of a length corresponding to the number of filters. The dimensions of the concatenated input H are [X, Y, Z]. In this way, information from all the residual routes 1 to M is retained in the concatenated input H. More particularly, information from the filters from each of the residual routes 1 to M is collected in the concatenated input H.

At step 606, the tensor manipulation unit 106C generates a first transposed input tensor H^{T} by transposing the concatenated input H to a form suitable for tensor multiplication with the first weight tensor W₁. In particular, the tensor manipulation unit 106C transposes the concatenated input such that the first and second dimensions are switched, and the third and second dimensions are switched. Therefore, the dimensions of the first transposed input tensor H^{T} are [Z, X, Y].

At step 608, the tensor multiplication unit 106D generates a first intermediate tensor by multiplying the first transposed input tensor H^{T} by the first weight tensor W₁. Thus, the first intermediate tensor is H^{T}W₁. The weights in the first weight tensor W₁ are learned to increase the contribution of some features (i.e., k-mers) over other features. The weights in the first weight tensor W₁ are optimised during a training process discussed further below.

At step 610, the activation function unit 106E generates a second intermediate tensor by applying the first activation function to the first intermediate tensor. In particular, the activation function unit 106E applies the tanh activation function, such that the second intermediate tensor is tanh (H^{T}W₁). The tanh activation function normalises the first intermediate tensor to values between −1 and 1 to avoid gradient explosion. It will be appreciated that any suitable activation function may be used to achieve this, but the tanh activation function has performed better empirically.

At step 612, the tensor multiplication unit 106D generates a third intermediate tensor by multiplying the second intermediate tensor by the second weight tensor W₂. Thus, the third intermediate tensor is tanh(H^{T}W₁)W₂. The weights in the second weight tensor W₂ are learned to increase the contribution of some features (i.e., k-mers) over other features. The weights in the second weight tensor W₂ are optimised during a training process discussed further below.

This step may be unnecessary but adds depth to the self-attention layer 106 that may improve learning capacity.

At step 614, the tensor manipulation unit 106C re-shapes the third intermediate tensor to a form suitable for input to the SoftMax activation function. In particular, the tensor manipulation unit 106C re-shapes the third intermediate tensor such that a first axis corresponds to the number of filters fᵢ, and a second axis comprises a concatenation of each spatial row from the concatenated input tensor for a given filter index along the first axis, with the x index prioritised.

Thus, the reshaped third intermediate tensor is an image constructed as follows:

$$[ {f}_{0} {x}_{0} {y}_{0} , {f}_{0} {x}_{0} {y}_{1} , … , {f}_{0} {x}_{0} {y}_{n} , {f}_{0} {x}_{1} {y}_{0} , {f}_{0} {x}_{1} {y}_{1} , … , {f}_{0} {x}_{1} {y}_{n} , … {f}_{0} {x}_{m} {y}_{0} , {f}_{0} {x}_{m} {y}_{1} , … {f}_{0} {x}_{m} {y}_{n} ] \\ [ {f}_{1} {x}_{0} {y}_{0} , {f}_{1} {x}_{0} {y}_{1} , … , {f}_{1} {x}_{0} {y}_{n} , {f}_{1} {x}_{1} {y}_{0} , {f}_{1} {x}_{1} {y}_{1} , … , {f}_{1} {x}_{1} {y}_{n} , … {f}_{1} {x}_{m} {y}_{0} , {f}_{1} {x}_{m} {y}_{1} , … {f}_{1} {x}_{m} {y}_{n} ] \\ [ … … … … … … … … … … … … … … … … … … … … … … … … … … … … … … … … ] \\ [ {f}_{g} {x}_{0} {y}_{0} , {f}_{g} {x}_{0} {y}_{1} , … , {f}_{g} {x}_{0} {y}_{n} , {f}_{g} {x}_{1} {y}_{0} , {f}_{g} {x}_{1} {y}_{1} , … , {f}_{g} {x}_{1} {y}_{n} , … {f}_{g} {x}_{m} {y}_{0} , {f}_{g} {x}_{m} {y}_{1} , … {f}_{g} {x}_{m} {y}_{n} ]$$

Wherein g is the number of filters, m is the length of the x dimension of the third intermediate tensor, and n is the length of the y dimension of the third intermediate tensor. Thus, the dimensions of the reshaped third intermediate tensor is [g, (x×m)]. The reshaped tensor will hereinafter be denoted as H′.

At step 616, the activation function unit 106E generates a fourth intermediate tensor by applying a second activation function to the re-shaped third intermediate tensor. In particular, the activation function unit 106E applies the SoftMax activation function, such that the fourth intermediate tensor is softmax(H′). Notably, the SoftMax activation function is applied along the filter axis to produce attention values, which indicate relative importance of each spatial position in the resultant tensor, for each filter set.

The filters from each residual route 1 to M are learnt independently of other routes. That is, a first filter of residual route 1 is stacked with filter 1 of route 2 and so on, but filter i in route j is learnt independently of filter i in all other routes. This combination is therefore arbitrary, and in theory, one may create a cross matrix with every possible combination of all filters in each route with all filters in every other route. However, such a cross matrix may grow to arbitrary computational complexity. Accordingly, stacking each filter index with its counterparts in other residual routes may reduce computational complexity, and the relative attention values are therefore only computed in these terms.

At step 618, the tensor manipulation unit 106C generates an attention tensor by reshaping the fourth intermediate tensor. The tensor is reshaped such that the dimensions of the attention tensor match the dimensions of the third intermediate tensor. Each concatenated element of the reshaped third intermediate tensor is converted to a corresponding position in a 3D tensor. For example, element f₀x₀y₀ of the reshaped third intermediate tensor is converted to element f₀,x₀,y₀. The attention tensor has dimensions [Z, X, Y]. Thus, the attention tensor has a first dimension corresponding to the filters of the residual blocks, a second dimension corresponding to the X dimension of the concatenated input tensor, and a third dimension corresponding to the Y dimension of the concatenated input tensor.

The attention tensor comprises a plurality of tension weights and is configured to weight the concatenated input matrix, as discussed in the following steps. The weighting provides a way of increasing and/or reducing the influence of different features (i.e., k-mers) to obtain the final output of the neural network.

At step 620, the tensor manipulation unit 106C generates a second transposed input tensor by transposing the concatenated input. The concatenated input is transposed such that the third dimension is swapped with the first dimension. The dimensions of the second transposed input tensor are thus [Z, Y, X].

At step 622, the tensor multiplication unit 106D generates an attention output by multiplying the second transposed input tensor by the attention tensor. The second transposed input tensor has dimensions [Z, Y, X], whilst the attention tensor has dimensions [Z, X, Y].

At step 624, the tensor manipulation unit 106C generates an output tensor OT by transposing the attention output. The attention output is transposed to have dimensions [Y, Z, X].

The attention weights are used to weight the different outputs of the residual routes 1 to M. Thus, k-mers of different length are effectively assigned differing levels of importance.

The self-attention layer 106 is a superior alternative to just concatenating the outputs of the residual routes together. The self-attention layer 106 allows the neural network to effectively weight (i.e., pay more attention) to those features (or combinations of features) from all residual route outputs that are more important for characterisation of the input genomic sequence. Without the self-attention layer 106, each output feature map from each residual route would have an equal weighting over the result, regardless of the overall set of extracted features.

This lack of feature preference can lead to diminished performance. Thus, the self-attention layer 106 provides an improved characterisation performance.

### Output Layer

The genome characterisation system 100 comprises an output layer 108. The output layer 108 is adapted to receive the output of the self-attention layer 106.

The output layer 108 is arranged to flatten the output of the self-attention layer 106. More particularly, the output layer 108 is arranged to flatten the output tensor OT. Flattening the output tensor OT involves converting the 3D output tensor OT to a 1D array by flattening a first 2D array of the 3D output tensor OT to a first portion of the 1D array, flattening a second 2D array of the output tensor OT to a second portion of the 1D array, and so on.

The 1D array is input to a densely connected layer connected to a sigmoid activation function layer, such that the output layer 108 provides an output vector having values between 0 and 1 corresponding to a plurality of characteristics.

In a simplified example, with example labels coronaviridae, respiratory symptoms, and haemorrhagic. The final output of the output layer 108 is a vector of length equal to the number of labels. Thus, in this example, the vector is of length 3. If the output vector is [0.9, 0.9, 0.1], then the output vector indicates a 90% confidence that the input genomic sequence is in the coronaviridae family, 90% confidence that the input genomic sequence has respiratory symptoms, and 10% confidence that the input genomic sequence is haemorrhagic.

A further step may comprise converting the neural network output to a characterisation output. This conversion step comprises assigning a plurality of labels based on the output vector. The number of labels is equal to the length of the output vector, wherein each entry of the output vector corresponds to a respective label. Assignment of labels is based on the confidence scores of the output vector. If an entry of the output vector meets a threshold, a respective entry of the characterisation output is ‘True.’ Continuing with the example, with the threshold is 0.8, the output of the system 100 is [Coronaviridae: True; Respiratory Symptoms: True; Haemorrhagic: True].

Thus, the genome characterisation system 100 outputs a set of labels that characterise the input genomic sequence, more particularly a genome of origin associated with the input genomic sequence.

### Genome Characterisation Method

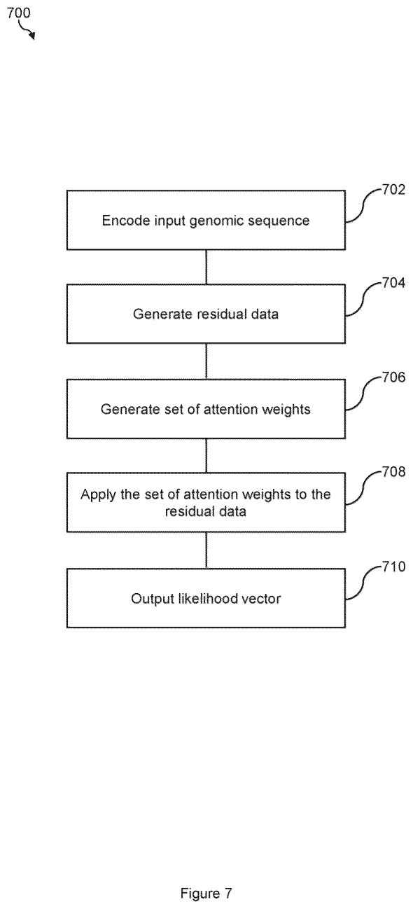

FIG. 7 shows a genome characterisation method 700 for providing a genome characteristic prediction of a genome of origin associated with an input genomic sequence using the genome characterisation system 100 of the present invention.

The genome characterisation method comprises: encoding (702) the input genomic sequence in a form suitable for input to a convolutional neural network; generating (704) residual data corresponding to features of differing length; generating (706) a set of attention weights based on the residual data and a set of weights; applying (708) the set of attention weights to the residual data to generate an attention output comprising data indicative of a relative importance of one or more portions of the input genomic sequence; and outputting (710) a likelihood vector indicative of characteristics of the genome.

The encoding (702) step is conducted by the input preparation layer 102. The generating (704) step is conducted by the multi-path residual block 104. The generating (706) and applying (708) steps are conducted by the self-attention layer 106. The outputting (710) step is conducted by the output layer 108.

### Neural Network Training

A trained model may be implemented using the application of an artificial neural network (ANN) or a convolutional neural network (CNN). CNNs can be hardware-based (neurons are represented by physical components) or software-based (computer models) and can use a variety of topologies and learning algorithms. CNNs usually have at least three layers that are interconnected. The first channel consists of input neurons arranged to receive input data.

Those neurons send data on to the second channel, referred to as a hidden layer, which implements a function and which in turn sends the output neurons to the third layer. There may be a plurality of hidden layers in the CNN. With respect to the number of neurons in the input layer, this parameter is based on training data.

The second or hidden layer in a neural network implements one or more functions. For example, the function or functions may each compute a linear transformation or a classification of the previous layer or compute logical functions. For instance, considering that the input vector can be represented as x, the hidden layer functions as h and the output as y, then the CNN may be understood as implementing a function f using the second or hidden layer that maps from x to h and another function g that maps from h to y. So, the hidden layer's activation is f(x), and the output of the network is g(f(x)).

CNNs can also be hardware or software based and can also use a variety of topologies and learning algorithms. A CNN usually comprises at least one convolutional layer where a feature map is generated by the application of a kernel matrix to an input image. This is followed by at least one pooling layer and a fully connected layer, which deploys a multilayer perceptron which comprises at least an input layer, at least one hidden layer and an output layer. The at least one hidden layer applies weights to the output of the pooling layer to determine an output prediction.

The training of the neural network may be implemented using any suitable approach. The weights and biases of the neural network are initialised with random values. A training dataset is prepared as discussed below, and input to the neural network to obtain a final output. The output is compared to ground truth values using a loss function, as discussed further below.

The parameters (i.e., weights and biases) of the neural network are updated by backpropagation, by calculating a gradient of the loss function and with respect to each weight by using the chain rule (i.e., gradient descent). The process is repeated for many iterations until a training threshold is met (e.g., the loss function no longer decreases significantly, a threshold number of iterations is met and/or the performance of the neural network begins to worsen). Further techniques such as regularisation to prevent overfitting may be employed.

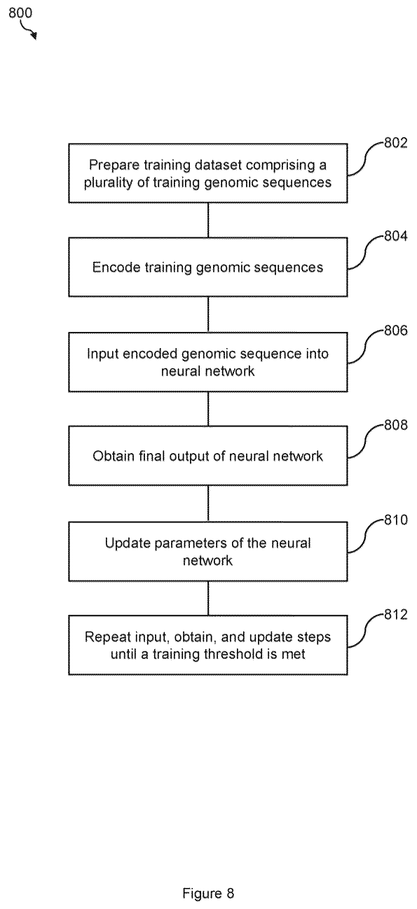

FIG. 8 shows a flow diagram of a method 800 for training the genome characterisation neural network of the genome characterisation system 100 of the present invention.

In a first step 802, the method 800 comprises preparing a training dataset comprising a plurality of genomic sequences. Preparing the training dataset comprises: obtaining reference genomes of a plurality of species; extracting one or more genomic sequences from each reference genome; modifying each of the genomic sequences; and labelling each modified genomic sequence with characteristic labels of the respective reference genome.

The species are viral pathogens, viral non-pathogens, and non-viral species. The characteristic labels include viral family host animal associations, symptoms, and tissue tropisms. These characteristics labels are in the form of a set of binary labels. For example, bovid carrier=True/False, Haemorrhagic=True/False, and other binary characteristics labels. Some species may be understudied and as such, dependable or definitive True/False distinctions for every label may not be obtainable. Accordingly, some labels may comprise an “unknown” label. An illustrative example comprises three labels—coronaviridae, respiratory symptoms, and haemorrhagic. An example reference genome may comprise ground truth labels [coronaviridae: True, respiratory symptoms: True, haemorrhagic: False].

To determine the reference genome, a publicly available database, such as NCBI RefSeq, may be consulted.

Extracting one or more genomic sequences from each reference genome comprises extracting one or more slices of the reference genome, each slice being a genomic sequence having a sequence length. The sequence length is preferably in line with what is expected from a real-world gene sequencer (for example, 800 to several thousand base pairs in length).

Modifying each genomic sequence comprises applying insertions, deletions, and/or base flips to the genomic sequence. Applying insertions to the genomic sequence comprises inserting one or more nucleotides into the genomic sequence. Applying deletions to the genomic sequence comprises deleting one or more nucleotides from the genomic sequence. Applying base flips to the genomic sequence comprises changing one or more nucleotides in the genomic sequence. Application of these insertions, deletions, and base flips is preferably in proportions that substantially match real world data, to replicate real-world applications of the model more accurately.

Step 804 comprises encoding the training genomic sequences in a form suitable for input to a convolutional neural network. In particular, the training genomic sequences input to the input preparation layer 102, which encodes the modified genomic sequences in a form suitable for input to the convolution neural network architecture (i.e., the multi-channel representation of the input genomic sequence). The encoded genomic sequences are input to the rest of the neural network as features, and the labels assigned to the parent genome of each genomic sequence are used as training targets.

Step 806 comprises inputting an encoded genomic sequence to the neural network. The encoded genomic sequence is in a form suitable for processing by the convolutional neural network.

Step 808 comprises obtaining a final output of the neural network. The final output of the neural network is a vector of length n, wherein n is the number of labels. Continuing with the example of three labels (coronaviridae, respiratory symptoms, and haemorrhagic) the final output could be a vector of length 3, [0.9, 0.9, 0.1]. This output vector indicates a 90% confidence that the input genomic sequence is in the coronaviridae family, 90% confidence that the input genomic sequence has respiratory symptoms, and 10% confidence that the input genomic sequence is haemorrhagic.

Step 810 comprises updating parameters of the neural network based on the final output. Updating the parameters of the neural network comprises comparing the final output of the neural network to ground truth values using a modified loss function. The ground truth values are 1 for True and 0 for false. Continuing with the example where the final output is [0.9, 0.9, 0.1] and the ground truth values are [1, 1, 0](Coronaviridae: True; Respiratory Symptoms: True; Haemorrhagic: True). The modified loss function is a modified form of the mean-squared-error loss function, as is known in the art. The modified loss function is arranged to mask or “ignore” labels which have been assigned as “unknown” in the ground-truth data. Accordingly, the computed gradient for each training iteration of the neural network is dependent on labels for which True/False labels are available.

Step 812 comprises repeating steps 806 to 810 until a training threshold is met. The training threshold could be any stop condition as is known in the art. For example, the training threshold could be a threshold loss function reduction, such that when the loss function no longer decreases significantly, the training process stops. Alternatively, the training threshold could be a threshold number of iterations.

The description provided herein may be directed to specific implementations. It should be understood that the discussion provided herein is provided for the purpose of enabling a person with ordinary skill in the art to make and use any subject matter defined herein by the subject matter of the claims.

It should be intended that the subject matter of the claims is not limited to the implementations and illustrations provided herein but include modified forms of those implementations including portions of implementations and combinations of elements of different implementations in accordance with the claims. It should be appreciated that in the development of any such implementation, as in any engineering or design project, numerous implementation-specific decisions should be made to achieve a developers' specific goals, such as compliance with system-related and business-related constraints, which may vary from one implementation to another. Moreover, it should be appreciated that such a development effort may be complex and time consuming, but would nevertheless be a routine undertaking of design, fabrication, and manufacture for those of ordinary skill having benefit of this disclosure.

Reference has been made in detail to various implementations, examples of which are illustrated in the accompanying drawings and figures. In the detailed description, numerous specific details are set forth to provide a thorough understanding of the disclosure provided herein. However, the disclosure provided herein may be practiced without these specific details. In some other instances, well-known methods, procedures, components, circuits, and networks have not been described in detail so as not to unnecessarily obscure details of the embodiments.

It should also be understood that, although the terms first, second, etc. may be used herein to describe various elements, these elements should not be limited by these terms. These terms are only used to distinguish one element from another. For example, a first element could be termed a second element, and, similarly, a second element could be termed a first element.

The first element and the second element are both elements, respectively, but they are not to be considered the same element.

The terminology used in the description of the disclosure provided herein is for the purpose of describing particular implementations and is not intended to limit the disclosure provided herein. As used in the description of the disclosure provided herein and appended claims, the singular forms “a,” “an,” and “the” are intended to include the plural forms as well, unless the context clearly indicates otherwise. The term “and/or” as used herein refers to and encompasses any and all possible combinations of one or more of the associated listed items. The terms “includes,” “including,” “comprises,” and/or “comprising,” when used in this specification, specify a presence of stated features, integers, steps, operations, elements, and/or components, but do not preclude the presence or addition of one or more other features, integers, steps, operations, elements, components and/or groups thereof.

While the foregoing is directed to implementations of various techniques described herein, other, and further implementations may be devised in accordance with the disclosure herein, which may be determined by the claims that follow. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A genome characterisation system for providing a genome characteristic prediction of a genome of origin associated with an input genomic sequence, the genome characterisation system comprising:
an input preparation layer arranged to encode the input genomic sequence in a form suitable for input to a convolutional neural network;
a multi-path residual block comprising a plurality of parallel residual routes, each residual route being adapted to receive input data from the input preparation layer and generate residual data corresponding to features of differing length;
a self-attention layer arranged to receive residual data from each of the residual routes, generate a set of attention weights based on the residual data and a set of weights, and apply the set of attention weights to the residual data to generate an output tensor comprising data indicative of a relative importance of one or more portions of the input genomic sequence; and
an output layer arranged to receive the output tensor from the self-attention layer; and output a likelihood vector indicative of characteristics of the genome of origin.

2. The genome characterisation system of claim 1, wherein the input preparation layer is arranged to encode the input genomic sequence as a multi-channel representation of the input genomic sequence.

3. The genome characterisation system of claim 2, wherein the input preparation layer is arranged to encode the input genomic sequence as the multi-channel representation by:
receiving the input genomic sequence;
generating a first channel comprising a frequency chaos game representation (FCGR) of the input genomic sequence;
generating a second channel representing a syntactic sequential relationship between nucleotide encodings in the first channel; and
encoding the input genomic sequence by combining the first and second channels to create the multi-channel representation of the input genomic sequence.

4. The genome characterisation system of claim 2, wherein the input preparation layer is further arranged to:
generate an input tensor representative of the input genomic sequence.

5. The genome characterisation system of claim 4, wherein the input tensor is a three-dimensional tensor comprising:
a height and width representative of the spatial aspect of the encoded genomic sequence; and
a depth representative of the multi-channel aspect of the encoded genomic sequence.

6. The genome characterisation system of claim 1, wherein each residual route comprises one or more residual blocks connected in sequence, each residual block comprising convolutional layers; wherein each convolutional layer of a residual route is of the same kernel size; and wherein the kernel size of convolutional layers in a first residual route is different to the kernel size of convolutional layers in a second residual route.

7. The genome characterisation system of claim 1, wherein the self-attention layer is arranged to:
generate a concatenated input by concatenating the input data from the plurality of residual routes;
generate a first transposed input tensor by transposing the concatenated input;
generate a first intermediate tensor by multiplying the first transposed input tensor by a first weight tensor;
generate a second intermediate tensor by applying a first activation function to the first intermediate tensor;
generate a third intermediate tensor by multiplying the second intermediate tensor by a second weight tensor;
re-shape the third intermediate tensor to a form suitable for input to a second activation function;
generate a fourth intermediate tensor by applying the second activation function to the re-shaped third intermediate tensor;
generate an attention tensor comprising the set of attention weights by reshaping a fourth intermediate tensor;
generate a second transposed input tensor by transposing the concatenated input;
generate an attention output by multiplying the second transposed input tensor by the attention tensor; and
generate an output tensor by transposing the attention output.

8. The genome characterisation system of claim 1, wherein the output layer is arranged to:
flatten the output tensor of the self-attention layer;
input the flattened tensor to a densely connected layer; and
apply a dense sigmoid activation function.

9. A genome characterisation method for providing a genome characteristic prediction of a genome of origin associated with an input genomic sequence, the genome characterisation method comprising:
encoding the input genomic sequence in a form suitable for input to a convolutional neural network;
generating residual data corresponding to features of differing length;
generating a set of attention weights based on the residual data and a set of weights, applying the set of attention weights to the residual data to generate an attention output comprising data indicative of a relative importance of one or more portions of the input genomic sequence; and
outputting a likelihood vector indicative of characteristics of the genome of origin.

10. A genomic sequence encoding method comprising:
receiving an input genomic sequence, the input genomic sequence comprising a one-dimensional linear sequence of nucleotide bases representing a particular genome; and
encoding the input genomic sequence in a form suitable for input to a convolutional neural network.

11. The method of claim 10, wherein the input genomic sequence is encoded as a multi-channel representation of the input genomic sequence.

12. The method of claim 11, wherein encoding the input genomic sequence as the multi-channel representation comprises:
receiving the input genomic sequence;
generating a first channel comprising a frequency chaos game representation (FCGR) of the input genomic sequence;
generating a second channel representing a syntactic sequential relationship between nucleotide encodings in the first channel; and
encoding the input genomic sequence by combining the first and second channels to create the multi-channel representation of the input genomic sequence.

13. A method for training a genome characterisation neural network comprising:
preparing a training dataset comprising a plurality of training genomic sequences;
encoding the training genomic sequences in a form suitable for input to a convolutional neural network;
inputting an encoded genomic sequence into the neural network;
obtaining a final output of the neural network;
updating parameters of the neural network based on the final output; and
repeating the inputting, obtaining, and updating steps until a training threshold is met.

14. The method of claim 13, wherein preparing the training dataset comprises:
obtaining reference genomes of a plurality of species;
extracting one or more genomic sequences from each reference genome;
modifying each of the genomic sequences; and
labelling each modified genomic sequence with characteristic labels of the respective reference genome.

15. The method of claim 14, wherein extracting one or more genomic sequences from each reference genome comprises:
extracting one or more slices of the reference genome, each slice being a genomic sequence having a sequence length.

16. The method of claim 14, wherein modifying each genomic sequence comprises applying insertions, deletions and/or base flips to the genomic sequence.

17. The method of claim 13, wherein encoding the training genomic sequences in a form suitable for to the convolutional neural network comprises:
generating a first channel comprising a frequency chaos game representation of the input genomic sequence;
generating a second channel representing a syntactic sequential relationship between nucleotides in the first channel; and
encoding the input genomic sequence by combining the first and second channels to create a multi-channel representation.

18. The method of claim 13, wherein the final output of the neural network is a vector of length n, wherein n is the number of labels.

19. The method of claim 13, wherein updating parameters of the neural network based on the final output comprises:
comparing the final output of the neural network to ground truth values using a modified loss function.

20. A neural network for providing a genome characteristic prediction of a genome of origin associated with an input genomic sequence, the neural network comprising:
a multi-path residual block comprising a plurality of parallel residual routes, each residual route being adapted to receive input data and generate residual data corresponding to features of differing length;
a self-attention layer arranged to receive residual data from each of the residual routes, generate a set of attention weights based on the residual data and a set of weights, and apply the set of attention weights to the residual data to generate an attention output comprising data indicative of a relative importance of one or more portions of the input genomic sequence; and
an output layer arranged to receive an output from the self-attention layer; and output a likelihood vector indicative of characteristics of the genome of origin.

21. The neural network of claim 20, wherein the input data is a three-dimensional tensor comprising:
a height and width representative of the spatial aspect of an encoded genomic sequence; and
a depth representative of a multi-channel aspect of the encoded genomic sequence.

22. The neural network of claim 20, wherein each residual route comprises one or more residual blocks connected in sequence, each residual block comprising convolutional layers; wherein each convolutional layer of a residual route is of the same kernel size; and wherein the kernel size of convolutional layers in a first residual route is different to the kernel size of convolutional layers in a second residual route.

23. The neural network of claim 20, wherein the self-attention layer is arranged to:
generate a concatenated input by concatenating the input data from the plurality of residual routes;
generate a first transposed input tensor by transposing the concatenated input;
generate a first intermediate tensor by multiplying the first transposed input tensor by a first weight tensor;
generate a second intermediate tensor by applying a first activation function to the first intermediate tensor;
generate a third intermediate tensor by multiplying the second intermediate tensor by a second weight tensor;
re-shape the third intermediate tensor to a form suitable for input to a second activation function;
generate a fourth intermediate tensor by applying the second activation function to the re-shaped third intermediate tensor;
generate an attention tensor comprising the set of attention weights by reshaping a fourth intermediate tensor;
generate a second transposed input tensor by transposing the concatenated input;
generate an attention output by multiplying the second transposed input tensor by the attention tensor; and
generate an output tensor by transposing the attention output.

24. The neural network of claim 20, wherein the output layer is arranged to:
flatten the output tensor of the self-attention layer;
input the flattened tensor to a densely connected layer; and
apply a dense sigmoid activation function.
